(19) United States
(12) Patent Application Publication
(10) Pub. No.: US 2003/0094501 A1
(43) Pub. Date: May 22, 2003

(54) Apparatus with potentiometer for dispensing an air freshener or insecticide

(76) Inventors: Shaun Patrick Rymer, East Yorkshire (GB); Clare Louise Robinson, Hull (GB)

Correspondence Address: FISH & RICHARDSON P.C., 45 ROCKEFELLER PLAZA, SUITE 2800, NEW YORK, NY, 10111, US

(21) Appl. No.: 09/989,616

(22) Filed: Nov. 20, 2001

(51) Int. Cl. A61L 9/04

(52) U.S. Cl. 239/6; 239/34; 239/57; 239/135

(57) ABSTRACT

An air freshener or insecticide dispenser apparatus comprises a reservoir, an electrochemical cell for generating a gas, and varying its rate of release. The gas generated acts on the air freshener or insecticidal composition to dispense said composition from the apparatus.

The present invention relates to an apparatus for dispensing an air freshener or insecticidal composition in the vapour phase.

Various devices for dispensing air freshener and insecticidal compositions are known. For example, the composition may be impregnated onto a solid carrier, from which it gradually evaporates and enters the atmosphere. Alternatively, the composition may be provided in the form of a liquid, for example, as a thickened liquid or gel. Such liquids are generally contained in a reservoir, formed, at least in part, of a material that is permeable to the vapours of the composition. As the liquid evaporates, the vapours of the composition can permeate through the material and enter the surrounding atmosphere.

WO 96/41159 describes an electrochemical cell for generating a gas for the transportation of fluid media. Specifically, the electrochemical cell of WO 96/41159 is designed to produce a gas at a controlled rate to actuate a piston or similar means to push the fluid out of a dispenser.

A new apparatus for dispensing air freshener and insecticidal compositions has recently been developed. The apparatus includes an electrochemical cell for generating a gas. As more gas is produced, the pressure in the apparatus increases. This build-up of pressure exerts a force on the air freshener or insecticidal composition and pushes it out of its reservoir, and onto an emanating pad, from which it evaporates into the atmosphere.

Surprisingly, it has now been found that the gas pressure within the apparatus is highly susceptible to fluctuations in temperature. At low temperatures, for example, the gas generated by the electrochemical cell contracts according to Boyle's Law. Thus, air from outside the apparatus is drawn into the apparatus. As a result, the force exerted on the air freshener or insecticidal composition is insufficient to dispense the composition from its reservoir. This problem does not arise with dispensers, such as those described in WO 96/41159, in which the electrochemical cell is required to generate gas for actuating a piston. In such cases, the gas is produced almost instantaneously, to move the piston in a short period of time. Thus, the gas does not remain in the apparatus for a sufficiently long period of time for atmospheric temperature fluctuations to have a significant impact on the rate at which fluid is dispensed.

According to the present invention, there is provided an apparatus for dispensing an air freshener or an insecticidal composition into the atmosphere, the apparatus comprising:

a reservoir containing an air freshener or insecticidal composition;

means for generating a gas; and

means for varying the rate of release of gas;

wherein in use, gas generated by the means for generating a gas acts on the air freshener or insecticidal composition to dispense the composition from the apparatus.

Preferably, the means for generating a gas is an electrochemical cell. Such a cell may be used to generate a gas by connecting the anode and the cathode of the electrochemical cell in a closed external circuit. The rate of release of gas may be altered by varying the resistance of the closed external circuit, for example, by using a potentiometer.

An advantage of the preferred embodiment described above is that the current through the circuit can be increased when the ambient temperature is low. This results in an increase in the rate of gas production, which compensates for the drop in the gas pressure in the apparatus at low temperatures. Similarly, when the ambient temperature is high, the current through the circuit can be reduced to decrease the rate of gas production. In this way, the gas pressure in the apparatus is maintained at an appropriate level to ensure that the air freshener or insecticidal composition is dispensed at the desired rate, in spite of ambient temperature fluctuations.

Preferably, the means for varying the resistance in the circuit is a potentiometer. The potentiometer may be employed to vary the current through the circuit between 0.08 and 0.8 mA, preferably, 0.14 and 0.4 mA. The resistance of the circuit may be varied between 2 and 20 Ohms, preferably, 3 and 16 Ohms, and more preferably, 4 and 13 Ohms. Preferably, the resistance is selected to ensure that the rate of gas production is maintained at a desired rate of, for example, 0.001 to 0.5 ml per 24 hour period, preferably, 0.1 to 0.3 ml per 24 hour period.

The potentiometer may be operated manually, for example, by using suitable actuating means. Such actuating means may take the form of a lever(s), a button(s), a dial(s) or the like. In a preferred embodiment, the actuating means takes the form of a lever, which is slidably mounted within a longitudinal aperture. The aperture may be provided with indicating means for indicating an appropriate position for the lever according to the ambient temperature. For example, the aperture may be provided with a temperature scale. In operation, a user can position the lever at the appropriate temperature indication to ensure that the resistance through the circuit is such that the air freshener or insecticidal composition is released at the desired rate. In an alternative embodiment, the aperture may be provided with indications such as “high” and “low”, “hot” and “cold”, or “min” and “max”. A user can position the lever at or between these extremes, depending on the rate of release of air freshener or insecticidal composition required. It may also be possible simply to mark the aperture with sequential numbers, or with a colour scale, for example, of red to blue. It should be understood that in addition to levers, the indicating means described above can also be used in combination with other actuating means, such as dials, buttons and the like.

The apparatus may also be provided with sensing means for sensing the ambient temperature. For example, the apparatus may be provided with a thermometer. Thus, a user can adjust the resistance of the circuit according to the ambient temperature of the apparatus.

In an alternative embodiment, a thermistor is provided. The resistance of a thermistor changes according to the ambient temperature. Thus, an advantage of this embodiment is that the resistance through the circuit can be varied automatically, without manual input.

Where the resistance of the circuit is varied automatically in response to the ambient temperature, the apparatus may be provided with means for overriding the automatic function, allowing the resistance to be varied manually.

The apparatus may also be provided with an on-off function to allow the current through the circuit to be turned on and off as desired.

Any suitable electrochemical cell for generating a gas may be employed in the apparatus of the present invention. Examples of suitable cells are described in WO 96/41159.

Any suitable gas can be generated to transport the air freshener or insecticidal composition from the apparatus. Examples include carbon dioxide, oxygen and preferably, hydrogen.

The reservoir may take the form of, for example, a tubular housing. The reservoir may have a capacity of 5 to 20 ml, preferably, 10 to 15 ml, for example, 12 to 14 ml. In one embodiment, a reservoir having a capacity of 14.5 ml is employed. The reservoir may be filled with 3 to 18 ml, preferably, 10 to 12 ml of air freshener or insecticidal composition.

The reservoir may be provided with at least one opening, from which the air freshener or insecticidal composition is allowed to escape. Preferably, a plurality of openings are provided. The total cross-sectional area of the opening(s) may be 0.01 to 1.2 mm², preferably, 0.1 to 0.5 mm², for example, 0.1 to 0.3 mm². Each opening may be defined by a micro-channel, which may or may not be substantially circular in cross section. The cross-sectional area of each micro-channel may be 0.01 to 0.4 mm², preferably, 0.05 to 0.1 mm², more preferably, 0.07 to 0.1 mm². In a preferred embodiment, 1 to 7, preferably, 2 to 5, for example, 3 openings are provided. In order to prevent the reservoir from releasing the air freshener or insecticidal composition prematurely, the opening or openings of the reservoir may be provided with a seal or cover, which may be ruptured or removed when the apparatus is ready for use.

In one embodiment, the opening or openings of the reservoir are located at one end of the reservoir. Thus, the electrochemical cell may be positioned at or adjacent the opposite end of the reservoir. In use, the gas generated by the cell can act on the air freshener or insecticidal composition and push it towards the other end of the reservoir, and out through the opening(s). Preferably, the gas acts directly on the composition. Thus, in such an embodiment, intervening members such as pistons or diaphragms are not required to push the composition out of the apparatus.

The apparatus may further comprise a protective casing to protect at least one of the components of the apparatus from damage. In one embodiment, the protective casing surrounds the reservoir. Preferably, the protective casing may also surround the electrochemical cell. The protective casing is generally provided with one or more apertures (eg vents) through which the vapours of the air freshener or insecticidal composition may flow into the atmosphere.

In a preferred embodiment, the apparatus is also provided with one or more emanators for aiding the dispersion of the air freshener or insecticidal composition into the atmosphere. In use, the air freshener or insecticidal composition emerging from the reservoir may drip onto the emanator, from which it evaporates into the atmosphere. The emanator may take the form of a membrane that is permeable to the air freshener or insecticidal composition. Such membranes may be formed of polyethylene or paper, and are generally thin, so as not to impede the flow of vapour into the atmosphere. For example, the membrane may have a thickness of 20 to 100 microns, preferably, 30 to 70 microns, and especially 40 to 60 microns. Suitable membranes are described in U.S. Pat. No. 4,634,614 and WO 98/23304.

In order to prevent the apparatus from releasing the air freshener or insecticidal composition before it is used, the membrane may be covered with an impermeable sheet that can be removed when the apparatus is ready for use. The sheet may be made of a laminate, such as polyethylene laminated with a metal such as aluminium.

The apparatus of the present invention is particularly suitable for dispensing an air freshener or insecticidal composition having a relative density of 0.5 to 1.2, preferably, 0.7 to 1.0, more preferably, 0.89 to 0.98. Preferably, also the air freshener or insecticidal composition has a flash point of 50 to 110 deg C., preferably, 60 to 95 deg C., more preferably, 65 to 88 deg C. Suitable air freshener and insecticidal compositions are described in WO 01/00304.

According to a further aspect of the present invention, there is provided a method for dispensing an air freshener or insecticidal composition into the atmosphere, the method comprising:

providing an apparatus as herein described;

actuating the means for generating a gas; and

allowing the gas generated to act on the air freshener or insecticidal composition in the reservoir to dispense the air freshener or insecticidal composition into the atmosphere.

In yet another aspect, the present invention provides the

use of means for varying the resistance in an external circuit formed by connecting the anode to the cathode of an electrochemical cell,

in an apparatus for dispensing an air freshener or an insecticidal composition,

to compensate for any undesired variations in the rate at which the air freshener or insecticidal composition is dispensed from the apparatus.

These and other aspects of the present invention will now be described by way of example with reference to the accompanying drawings, in which:

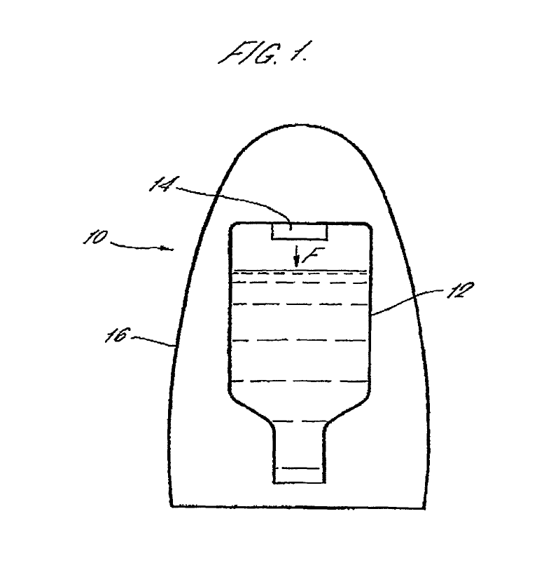

FIG. 1 is a schematic diagram of an apparatus according to a preferred embodiment of the present invention; and

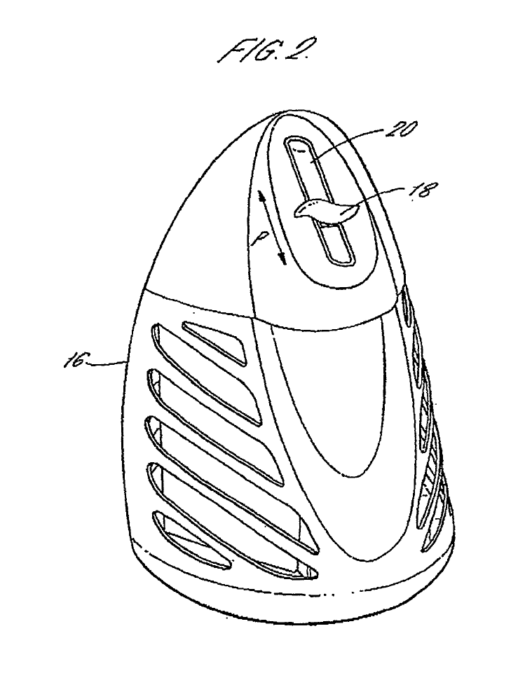

FIG. 2 is a schematic diagram of the exterior of the apparatus of FIG. 1.

Reference is made to FIG. 1 of the drawings, which depicts an apparatus 10 for dispensing an air freshener. The apparatus comprises a reservoir 12 containing an air freshener, and an electrochemical cell 14 for generating hydrogen. The electrochemical cell 14 is connected to a potentiometer (not shown) for varying the resistance of a closed external circuit formed by connecting the anode to the cathode of the electrochemical cell 14. The apparatus 10 also comprises an outer casing 16.

In use, the electrochemical cell 14 is actuated by connecting the anode of the cell 14 to the cathode. This causes hydrogen to be produced, which accumulates in the apparatus 10. As the gas pressure in the apparatus 10 increases, a downward force F is exerted on the air freshener in the reservoir 12. This downward force F pushes the air freshener out of the reservoir 12 and onto an emanating pad (not shown), from which it evaporates into the atmosphere.

Fluctuations in the surrounding temperature have a significant effect on the gas pressure within the apparatus 10. For example, when the ambient temperature falls, the gas pressure within the apparatus 10 is reduced. Thus, although the electrochemical cell 14 is producing hydrogen, the rate of gas production is too low to maintain the gas pressure at its optimum value. Accordingly, the gas pressure within the apparatus 10 is insufficient for air freshener to continue to be dispensed from the reservoir 12 at the appropriate rate.

To overcome this problem, the potentiometer can be actuated to reduce the resistance of the circuit. This results in an increase in current, which in turn, raises the rate of hydrogen production. This increase in hydrogen generation compensates for the drop in gas pressure brought about by the fall in temperature. In this manner, the hydrogen pressure with in the apparatus 10 can be restored to its optimum value, allowing air freshener to be dispensed from the reservoir 12 in the appropriate manner.

When the ambient temperature increases, the rate of hydrogen production can be reduced by increasing the resistance of the circuit using the potentiometer.

Reference is now made to FIG. 2, which depicts the outer casing 16 of the apparatus 10 in greater detail. The casing 16 is provided with a lever 18, which is slidably mounted in a longitudinal aperture 20. The lever 18 is coupled to the potentiometer and can be moved to alter its resistance. Thus, by sliding the lever along the aperture as indicated by arrows P, the resistance of the circuit can be altered to vary the rate of hydrogen generation.

We claim:

1. An apparatus for dispensing an air freshener or an insecticidal composition into the atmosphere, the apparatus comprising:
a reservoir containing an air freshener or insecticidal composition;
means for generating a gas; and
means for varying the rate of release of gas;
wherein, in use, gas generated by the means for generating a gas acts on the air freshener or insecticidal composition to dispense the composition from the apparatus.

2. An apparatus as claimed in claim 1, wherein the means for generating a gas is an electrochemical cell.

3. An apparatus as claimed in claim 2, wherein the means for varying the rate of release of gas comprises means for varying the resistance of a closed external circuit formed by connecting the anode and cathode of the electrochemical cell.

4. An apparatus as claimed in claim 3, wherein the means for varying the resistance is a potentiometer.

5. An apparatus as claimed in claim 4, wherein the potentiometer is employed to vary the current through the circuit between 0.08 to 0.8 mA.

6. An apparatus as claimed in claim 5, wherein the potentiometer is employed to vary the resistance of the circuit between 2 and 20 ohms.

7. An apparatus as claimed in claim 6, wherein the resistance is selected to ensure that the rate of gas production is maintained at from 0.001 to 0.5 ml per 24 hour period.

8. An apparatus as claimed in any one of claims 4, 6 or 7, wherein the means for varying the resistance is operated using an actuating means.

9 An apparatus as claimed in claim 8, wherein the actuating means is a lever, a button a dial.

10. An apparatus as claimed in claim 9, which further comprises indicating means for indicating an appropriate position for the actuating means depending on the ambient temperature.

11. An apparatus as claimed in claim 1, 4 or 7, which comprises sensing means for sensing the ambient temperature.

12. An apparatus as claimed in claims 4 or 7, wherein the potentiometer is a thermistor.

13. A method for dispensing an air freshener or insecticidal composition into the atmosphere, the method comprising:
providing an apparatus as claimed in claim 1;
actuating the means for generating a gas; and
allowing the gas generated to act on the air freshener or insecticidal composition in the reservoir to dispense the air freshener or insecticidal composition from the apparatus.

14. In an apparatus for dispensing an air freshener or an insecticidal composition, a method to compensate for any undesired variations in the rate at which the air freshener or insecticidal composition is dispensed from said apparatus, which method comprises varying the resistance in an external circuit formed by connecting the anode to the cathode of an electrochemical cell contained within said apparatus.
